(19) United States
(12) Patent Application Publication
(10) Pub. No.: US 2005/0154255 A1
(43) Pub. Date: Jul. 14, 2005

(54) Surgical device

(75) Inventor: Ian Jacobs, Moorestown, NJ (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: The Children's Hospital of Philadelphia, Philadelphia, PA (US)

(21) Appl. No.: 10/994,503

(22) Filed: Nov. 22, 2004

Related U.S. Application Data
(60) Provisional application No. 60/523,713, filed on Nov. 20, 2003.

(51) Int. Cl. A61B 18/04; A61B 1/00

(52) U.S. Cl. 600/104

(57) ABSTRACT

A surgical device used during a surgical procedure combines a cutting tool and an optical device for removing tissue from a distal area with visual inspection of the tissue that needs to be removed. In a preferred embodiment, the surgical device includes a cutting tool for removing tissue from an internal body part, the cutting tool including a cannula connected to a shaving member that cuts the tissue for removal, and an optical member coupled to the cutting tool for viewing the internal body part during a surgical proceeding.

### FIELD OF THE INVENTION

This invention relates to electrosurgical methods and apparatus for removing tissue from a target site of a patient.

### BACKGROUND OF THE INVENTION

Microdebriders have been widely utilized in the field of otolaryngology. A microdebrider is generally a powered rotary shaving device with continuous suction often used during endoscopic sinus surgery. Typical microdebriders include a cannula or tube, connected to a hand piece, which in turn is connected to a motor with a foot control and a suction device. Microdebriders have been used in performing surgical procedures, such as partial tonsillectomies, by partially shaving the tonsils. This procedure entails eliminating the obstructive portion of the tonsil while preserving the tensile capsule. A partial tonsillectomy results in less post-operative pain, a more rapid recovery, and perhaps fewer delayed complications than a full tonsillectomy. One example of the microdebrider is the XPS 3000 System manufactured by Medtronics/Xomed (Medtronics USA, Inc., Jacksonville Fla.) (see references 1-5).

Microdebriders are useful in performing tonsillectomy, in part because the surgeon can see the tonsils while performing the tonsillectomy. Unfortunately, a surgeon can not readily remove tissue from further down in the airway (e.g., the distill airway) because the tissue desired for removal can not be seen simply by looking into the mouth. Accordingly, surgeons use an optical device (e.g., a telescope) with a diode or light source to enable the surgeon to see farther down the airway. However, the use of the microdebrider is limited to areas visible to a surgeon performing the operation, such as for example tonsillectomy and adenoidectomy. Moreover, it is cumbersome to hold the optical device and microdebrider while performing surgery.

U.S. Pat. No. 6,589,237, to Woloszko et al discloses electrosurgical methods and an apparatus for removing tissue from a target site of a patient. An electrosurgical suction apparatus disclosed in the patent includes an aspiration channel in communication with a distal aspiration port, and an electrode support having a plurality of active electrodes disposed thereon, the plurality of active electrodes spanning the aspiration port. Each of the plurality of active electrodes is adapted for removing tissue from a target site to form low molecular weight ablation by-products. Each of the plurality of active electrodes is also adapted for digesting resected tissue fragments to yield low molecular weight ablation by-products. Ablation by-products and resected tissue fragments are readily removed from the target site via an aspiration stream flowing proximally through the aspiration channel. In one embodiment, each of a plurality of active electrodes protrude in different directions from a beveled treatment surface of the electrode support, and each of the plurality of active electrodes extend from the beveled treatment surface by substantially the same orthogonal distance.

The prior art procedure for the removal of papilomas includes laser surgery. Unfortunately, laser surgery can cause scarring of the vocal folds.

Using a microdebrider in distal airways is further complicated because of the impaired visibility. Providing light in distal airways by using, for example, an endoscope is one way of improving visibility. However, holding both instruments at the same time is very inconvenient for the surgeon.

Despite the foregoing developments, the inventor discovered that there is a need to provide a surgical device capable of performing a function of a microdebrider while providing visibility at the target site of a patient. It would be beneficial to provide an approach for removing tissue in a distal area with visual inspection of the tissue that needs to be removed.

All references cited herein are incorporated herein by reference in their entireties.

### BRIEF SUMMARY OF THE INVENTION

Accordingly, in a preferred embodiment, the invention provides a surgical device used during a surgical procedure. The surgical device includes a cutting tool and an optical member. The cutting tool is used for removing tissue from an internal body part and includes a cannula connected to a shaving member that cuts the tissue for removal by the cutting tool. The optical member is coupled to the cutting tool and is used for viewing the internal body part during a surgical proceeding. The cutting tool may also include an aspiration unit for withdrawing the cut tissue from the internal body part. The aspiration unit is adapted for coupling to a vacuum source and includes an aspiration channel terminating in a distal aspiration port.

In another preferred embodiment, the optical member includes a telescope. In yet another preferred embodiment, the cutting tool is a microdebrider. The invention further provides a surgical device including optical means for viewing an internal body part during a surgical proceeding, and cutting means for removing tissue from an internal body part. The cutting means is attached to the optical means for one handed operation.

The invention also provides a method for removing tissue from a distal airway of a patient, including inserting a surgical device having a cutting tool and an optical member coupled to the cutting tool through a patients mouth into the distal airway, viewing the tissue via the optical member, shaving the tissue from the distal airway with the cutting tool, and withdrawing the shaved tissue from the distal airway with the surgical device.

### BRIEF DESCRIPTION OF SEVERAL VIEWS OF THE DRAWINGS

The invention will be described in conjunction with the following drawings in which like reference numerals designate like elements and wherein:

FIGS. 1 and 2 are plan views of the surgical device in accordance with an exemplary embodiment of the invention showing a coupled microdebrider and endoscope.

### DESCRIPTION OF PREFERRED EMBODIMENTS

The invention was driven by the desire to develop a surgical device usable in areas not readily visible to a human eye.

The inventor started using a microdebrider for surgical removal of obstructions to distal airways, for example, removal of papillomas. Papillomatous lesions (also called respiratory papillomatosis) are wart-like growths that grow on the membrane surfaces of the larynx, which usually cause hoarseness. Papilloma lesions have a viral etiology and in some cases can grow large enough to interfere with breathing and thus be life threatening. Even through alternative methods (e.g., anti-viral medication) are sometimes employed, the main treatment for laryngeal papilloma continues to be surgical treatment.

One of the advantages of the preferred microdebrider discussed in greater detail below is its ability to precisely remove undesired tissue under direct visualization while leaving adjacent structures undisturbed. The present invention offers an improvement that allows even better precision especially in the areas where visibility is limited.

This invention solves problems in the prior art by combining a cutting tool (e.g., microdebrider) and an optical device (e.g., scope) in one device. A preferred microdebrider usable in the invention (e.g., an XPS 3000 System by Medtronics) includes of an outer windowed sheath surrounding an inner rotating hollow blade (or bur) which is connected to a continuous suction line. The window is directed by the surgeon toward the tissue to be removed which will be drawn in by the vacuum, and the rotating blade then shaves the tissue.

Procedures of removal an unwanted tissue from an organism may be performed with the preferred microdebrider through the mouth or nose using speculae or gags, or using endoscopic techniques, such as functional endoscopic sinus surgery (FESS). These procedures may include the removal of swollen tissue, chronically-diseased inflamed and hypertrophic mucus linings, polyps and/or neoplasms from the various anatomical sinuses of the skull, the turbinates and nasal passages, in the tonsil, adenoid, epi-glottic and supra-glottic regions, and salivary glands, submucus resection of the nasal septum, excision of diseased tissue and the like. In other procedures, the present invention may be useful for collagen shrinkage, ablation and/or hemostasis in procedures for treating snoring and obstructive sleep apnea (e.g., soft palate, such as the uvula, or tongue/pharynx stiffening, and midline glossectomies), for gross tissue removal, such as tonsillectomies, adenoidectomies, tracheal stenosis and vocal cord polyps and lesions, or for the resection or ablation of facial tumors or tumors within the mouth and pharynx, such as glossectomies, laryngectomies, acoustic neuroma procedures and nasal ablation procedures. In addition, the present invention is useful for procedures within the ear, such as stapedotomies, tympanostomies or the like.

The invention will be illustrated in more detail with reference to the following Examples, but it should be understood that the present invention is not deemed to be limited thereto. A surgeon using an exemplary surgical instrument with the coupled optical device and microdebrider according to the preferred embodiments can perform operations in the distal airway, including in the trachea region. The surgeon can also remove papilloma and other morbid or contaminated tissue from the distal airway as needed to benefit the patient.

A preferred embodiment of the invention is described in greater detail below and can be seen in the figures as a surgical device including an optical device for internal viewing of a patient coupled to a microdebrider. The optical device and debrider may be coupled by ways readily understood by a skilled artisan (e.g., an adapter, adhesive, bonding, fusing, tape, winding). By coupling the optical device to the midrodebrider, a surgeon can see much more effectively the target tissue and perform surgery on the tissue at the same time. Preferably, the optical unit includes a light source (e.g., diode) that brightens the target area of the distal pathway so that the surgeon can see the tissue that needs to be removed.

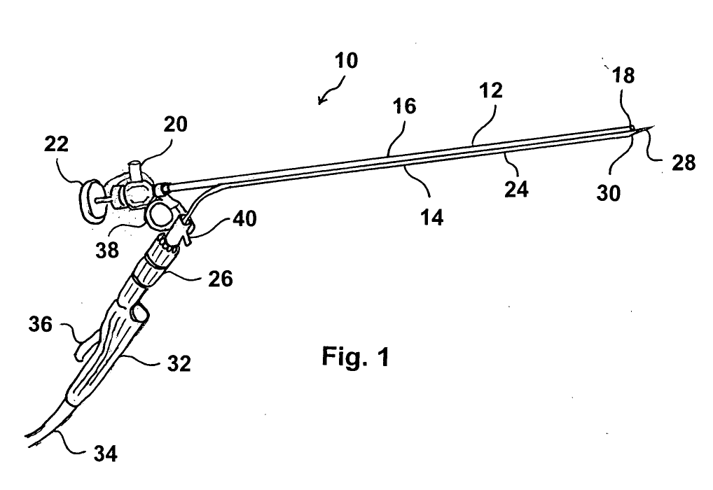

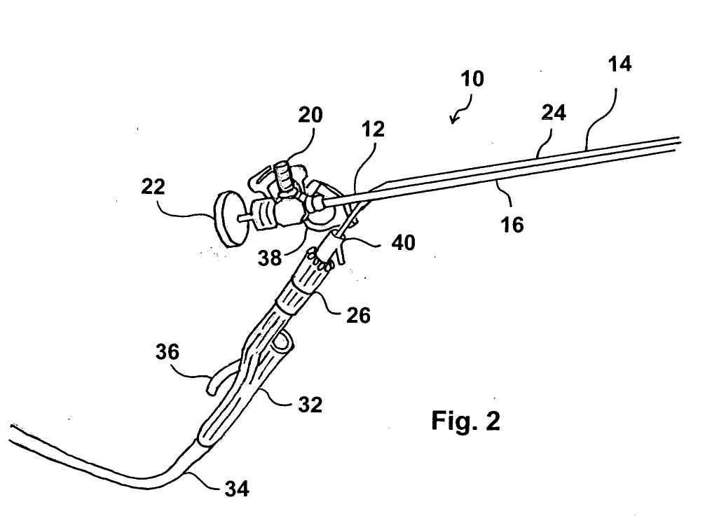

Exemplary microdebriders in accordance with a preferred embodiment of the invention are shown in the figures. In particular, FIGS. 1 and 2 illustrate a microdebrider apparatus 10 including a surgical optical device (e.g., scope, telescope 12) coupled to a midrodebrider 14. The telescope 12 and microdebrider 14 are coupled in such a way as to allow a surgeon to use both instruments as one combined instrument with one hand. The telescope 12 includes an elongated shaft 16 extending to a lens and diode 18 in one direction and a video connector 20 in the opposite direction. The telescope 12 may also include a knob 22 for adjusting the focus of the telescope 12, and for helping to secure the video connector 20 to the elongated shaft 16.

The microdebrider 14 includes an elongated cannula 24 that may be flexible or rigid, a handle 26 coupled to the proximal end of the cannula 24 and a shaving member 28 located at a distal end 30 of the cannula. The distal end 30 may be bladed, as shown in FIG. 1, but is not limited thereto. The handle 26 includes a connector housing 32 at its proximal end, which can be removably connected to a power supply cable 34. The power supply cable 34 couples the handle 26 to a power supply or motor for actuating the shaving member 28. The handle 26 also preferably includes a suction tube 36 that, in use, is coupled to a fluid removal element (not shown) for aspirating debris from the cutting area of the shaving member 28.

The microdebrider apparatus 10 also includes first and second gripping members 38 and 40 to help the surgeon control the microdebrider apparatus. The first gripping member 38 is coupled to both the telescope 12 and the microdebrider 14 near their proximal end, and includes apertures arranged to accept fingers of the surgeon for control during surgery. The second gripping member 40 is coupled to the microdebrider 14 adjacent the handle 26 and is arranged to engage the surgeon's finger for control. Preferably the gripping members are made of a plastic, a hard rubber or of a similar lightweight non-slippery material that helps the surgeon hold the microdebrider apparatus 10. While the gripping member 38 is preferably coupled to both the telescope 12 and the microdebrider 14, it is understood that the gripping member could be attached to either of the telescope or microdebrider as desired to aid in the holding and control of the microdebrider apparatus 10.

Both the telescope 12 and the microdebrider 14 have been used in the art for their intended purposes. However, the inventor discovered that a surgeon cannot readily and accurately remove tissue when the tissue desired for removal cannot be seen simply by looking into the body orifice open (e.g., mouth, nose, ear, etc.). The inventor also discovered that while an optical device with a diode or light source enables the surgeon to see farther down the passageway, the use of the microdebrider is limited to areas visible to a surgeon performing the operation, and it is cumbersome to hold the optical device and microdebrider while performing surgery. Accordingly, the inventor coupled the telescope 12 with the microdebrider 14 in such a way as to provide visibility to the surgeon at the target site while allowing the surgeon to simultaneously use both instruments with one hand.

As can best be seen FIG. 1, the telescope 12 preferably extends to the distal end of the microdebrider 14 but does not interfere with the shaving member 28 located at the distal tip of the microdebrider. That is, the diode 18 is adjacent but preferably set back from the shaving member 28 where it can display the target site before, during and after tissue is removed from the target site, and not interfere with the cutting. The scope 12 and microdebrider 14 are coupled along substantially the entire length of the cannula 24 from near the bladed distal end 30 to the handle 26. As noted above, the handle 26 of the microdebrider 14 includes a suction tube 36 that extends and attaches to a vacuum source (e.g., suction device). The video connector 20 located at the proximal end of the telescope 12, closest to the handle 26, sends signals to a video unit that displays the image taken by the telescope.

Wile not being limited to a particular theory, the cannula 24 is substantially straight along a majority of its length, bending at about a 45° angle as it approaches the handle 26. Moreover, the elongated shaft 16 of the telescope 12 is substantially straight along its length, and is coupled to the cannula 24 along their abutting relationship. However, it is understood that both the cannula 24 and the shaft 16 may be bent at various angles to improve access to the operation site of tissue being treated and depending on the procedure, while being well within the scope of the invention.

While the telescope 12 is described as being coupled to a debrider having a shaving member at its distal tip, it is understood that the scope of the invention is not limited to this embodiment. For example, the telescope may be coupled to other surgical instruments used for removing tissue, including instruments that use laser to cut tissue.

It is also understood that the microdebrider and telescope described and shown are exemplary indications of preferred embodiments of the invention, and are given by way of illustration only. In other words, the concept of the present invention may be readily applied to a variety of preferred embodiments, including those disclosed herein. While the invention has been described in detail and with reference to specific examples thereof, it will be apparent to one skilled in the art that various changes and modifications can be made therein without departing from the spirit and scope thereof.

1. A surgical device used during a surgical procedure, comprising:
a cutting tool for removing tissue from an internal body part, said cutting tool including a cannula connected to a shaving member that cuts the tissue for removal by the cutting tool; and
an optical member coupled to said cutting tool, said optical member for viewing the internal body part during the surgical procedure.

2. The surgical device of claim 1, wherein said cutting tool includes an aspiration unit for withdrawing the cut tissue from the internal body part, said aspiration unit adapted for coupling to a vacuum source, said aspiration unit including an aspiration channel terminating in a distal aspiration port.

3. The surgical device of claim 1, wherein said optical member includes a telescope.

4. The surgical device of claim 1, further comprising a light source coupled to said optical member for emitting light onto the internal body part.

5. The surgical device of claim 1, wherein the internal body part is in a distal airway region of a patient.

6. The surgical device of claim 5, wherein the internal body part is a trachea.

7. The surgical device of claim 1, wherein said cutting tool is a microdebrider.

8. The surgical device of claim 1, said optical member including a diode and a video connector communicating a video image from said diode to a video unit capable of displaying the image.

9. A surgical device, comprising:
optical means for viewing an internal body part during a surgical proceeding; and
cutting means for removing tissue from an internal body part, said cutting means attached to said optical means for one handed operation.

10. The surgical device of claim 9, wherein said cutting means includes aspiration means for withdrawing the cut tissue from the internal body part, said aspiration means adapted for coupling to a vacuum source, said aspiration means including an aspiration channel terminating in a distal aspiration port.

11. The surgical device of claim 9, wherein said optical means includes is a telescope.

12. The surgical device of claim 9, further comprising a light source coupled to said optical means for emitting light onto the internal body part.

13. The surgical device of claim 9, wherein the internal body part is in a distal airway region of a patient.

14. The surgical device of claim 9, wherein said cutting means includes a microdebrider.

15. The surgical device of claim 9, said optical means including a diode and a video connector communicating a video image from said diode to a video unit capable of displaying the image.

16. A method for removing tissue from a distal airway of a patient, comprising inserting a surgical device having a shaving member and an optical member coupled to the shaving member through a patients mouth into the distal airway, viewing the tissue via the optical member, shaving the tissue from the distal airway with the shaving member, and withdrawing the shaved tissue from the distal airway.
